SEQUENCE LISTING (2 sequences)

SEQ ID NO: 1 - DNA, 1419 bp, Homo sapiens
ccacgcgtccgagaccaaggagtggaaagttctccggcagccctgagatctcaagagtgacatttgtgagaccagctaatttgattaaaattctcttggaatcagctttgctagtatcatacctgtcgcagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtgaaatggaagtcaatagggctgttgggactttcttgaaaagaagcaaggaaatatgagtcatccgctatcacagctttcaaaagcaagaacaccatcctacataatacccaggattcccccaacacacgttcttttctaaatgccaatgagttggcctttaaaaatgcaccactttttttttttttttggacagggtctcactctgtcacccaggctggagtgcagtggcaccaccatggctctctgcagccttgacctctgggagctcaagtgatcctcctgcctcagtctcctgagtagctggaactacaaggaagggccaccacacctgactaacttttttgttttttgttggtaaagatggcatttcgccatgttgtacaggctggtctcaaactcctaggttcactttggcctcccaaagtgctgggattacagacatgaactgccaggcccggccaaaataatgcaccacttttaacagaacagacagatgaggacagagctggtgat

SEQ ID NO: 2 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

(19) United States
(12) Patent Application Publication
(10) Pub. No.: US 2006/0121030 A1
(43) Pub. Date: Jun. 8, 2006

(54) Use of cd 137 antagonists for the treatment of tumors

(76) Inventors: Herbert Schwarz, Kranzberg (DE); Margarethe Wittmann, Frankfurt/Main (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/539,257

(22) PCT Filed: Dec. 16, 2003
(86) PCT No.: PCT/EP03/14330

§ 371 Date: Jan. 30, 2006

(30) Foreign Application Priority Data
Dec. 16, 2002 (EP) 02028264.6

(51) Int. Cl. A61K 48/00; A61K 39/395

(52) U.S. Cl. 424/144.1; 424/93.2; 514/44

(57) ABSTRACT

The present invention relates to the therapy of tumors through neutralisation of CD137 or inhibition of CD137 expression of CD137 antagonising molecules. Furthermore, the present invention provides the use of CD137 or agonistic anti-CD307 ligand antibodies for the treatment of conditions characterised by overactive immune reactions.

The present invention relates to the therapy of tumors through neutralisation of CD137 or inhibition of CD137 expression by CD137 antagonising molecules. Furthermore, the present invention provides the use of CD137 or agonistic anti-CD137 ligand antibodies for the treatment of conditions characterised by overactive immune reactions.

Many tumor patients develop an immune response against their tumors. However, often this immune response is not sufficient for tumor eradication. As a consequence, tumor cells develop and are being selected for their successful development of defense mechanisms against the host Immune response. One category of these defense mechanisms is the ectopic expression of immunoregulatory molecules, which inhibit the host immune response. For example, many glioblastomas express TGF-β, a potent antiinflammatory molecule and the neutralisation of TGF-β can enable the immune system to eliminate the tumor Jachimczak et al., 1993). Another immune regulatory molecule is CD95 ligand, which is expressed by cytotoxic T cells and natural killer cells in order to induce programmed cell death in target cells. CD95 ligand can be expressed by normal tissues to maintain an immune privileged status. Its ectopic expression is exploited by hepatomas and other tumors which use CD95 ligand to kill tumor-infiltrating immune cells (Strand et al., 1996, O'Connel et al., 1999).

The cytokine receptor CD137 is a member of the tumor necrosis factor receptor family. CD137 is expressed by activated T and B lymphocytes and expression in primary cells is strictly activation dependent (Schwarz et al., 1995). The gene for human CD137 resides on chromosome 1p36, in a cluster of related genes, and this chromosomal region is associated with mutations in several malignancies (Schwarz et al., 1997).

Crosslinking of CD137 co-stimulates proliferation of T lymphocytes (Goodwin et al., 1993; Pollock et al., 1993; Schwarz et al., 1996), and CD137 ligand expressed by B lymphocytes co-stimulates T cell proliferation synergistically with B7 (DeBenedette et al., 1995).

While agonistic antibodies and the ligand to CD137 enhance lymphocyte activation, CD137 protein has the opposite effect. It inhibits proliferation of activated T lymphocytes and induces programmed cell death. These T cell-inhibitory activities of CD137 require immobilisation of the protein, arguing for transmission of a signal through the ligand/coreceptor (Schwarz et al., 1996; Michel et al., 1999).

The known human CD137 ligand is expressed constitutively by monocytes and its expression is inducible in T lymphocytes (Alderson et al., 1994). Monocytes are activated by immobilised CD137 protein and their survival is profoundly prolonged by CD137. (Langstein et al., 1998; Langstein et al., 1999a). CD137 also induces proliferation in peripheral monocytes (Langstein et al., 1999b). Macrophage colony-stimulating factor (M-CSF) is essential for the proliferative and survival-enhancing activities of CD137 (Langstein et al., 1999a; Langstein et al., 1999b).

Signalling through CD137 ligand has also been demonstrated in B cells where it enhances proliferation and immunoglobulin synthesis. This occurs at interactions of B cells with CD137-expressing T cells or follicular dendritic cells (Pauly et al., 2002). It was postulated that similarly to the CD40 receptor/ligand system, which mediates T cell help to B cells after first antigen encounter, the CD137 receptor/ligand system may mediate co-stimulation of B cells by FDC during affinity maturation (Pauly et al., 2002).

Soluble forms of CD137 are generated by differential splicing and are selectively expressed by activated T cells (Michel et al., 1998). Soluble CD137 is antagonistic to membrane-bound or immobilised CD137, and levels of soluble CD137 correlate with activation induced cell death in T cells (DeBenedette et al., 1995; Hurtado et al., 1995; Michel et al., 2000).

The problem underlying the present invention is to provide a novel system for the treatment of tumors which overcomes the defence mechanisms of tumor cells against the host immune system. A further object of the present invention is to provide a novel system for treating conditions characterised by overactive or undesired immune reactions.

The solution to the above problems is achieved by the embodiments of the present invention characterised in the claims.

According to a first aspect, the present invention provides the use of CD137 antagonists (or the use of CD137 antagonists for the preparation of a medicament or pharmaceutical composition) for the treatment of cancer, i.e. tumors expressing CD137.

The present invention is based on the finding that CD137 is expressed by tumors as a neoantigen and provides protection from the host immune response. Specifically, CD137 induces apoptosis in cytotoxic immune cells. In addition, CD137 expression leads to TGF-β secretion by the tumor cells which further inhibit anti-tumor immune responses.

Therefore, the neutralisation of CD137 expressed by tumors and/or inhibition of CD137 expression by tumors enables the immune system of a patient to eliminate or at least reduce the tumor mass.

The term “CD137 antagonist” refers to a chemical entity being capable of reducing or eliminating at least one function of CD137 or a functional analogue or equivalent thereof. The interference with a CD137 function can be exerted by any direct or indirect mechanism, including inhibition or neutralisation by binding of molecules, down regulation of CD137 expression, expression of non-functional CD137 derivatives, neutralisation or inhibition of CD137 ligands, in particular the natural CD137 ligand, as well as inhibition of CD137 ligand expression.

According to a preferred embodiment of the present invention, the CD137 antagonist is selected from CD137-specific antibodies, peptides, organic small molecules, antisense oligonuclotides, siRNAs, antisense expression vectors or recombinant viruses.

As already mentioned above, it is also possible to antagonise the function of CD137 via neutralisation or inhibition of CD137 ligand or by down regulating its expression. Therefore, the CD137 antagonist may be selected from CD137 ligand-specific antibodies, peptides, organic small molecules, antisense nucleic acids such as antisense oligonucleotides, siRNAs, antisense expression vectors or recombinant viruses as well.

Inhibitors of CD137 or CD137 ligands can bind to CD137 or to CD137 ligand via any chemical or physical interaction, including covalent binding, hydrogen bonds, electrostatic interactions and Van-der-Waals interactions. By way of any of the above interactions at least one function of CD137, in particular the inhibition of proliferation of immune cells, e.g. activated T lymphocytes, and induction of programmed cell death in such immune cells, is inhibited.

From a chemical point of view the CD137 antagonist useful in the context of the present invention comprises any chemical entity, in particular compounds which are generally suitable as medical drugs in tumor therapy. Examples of compounds useful as CD137 antagonists are organic small molecules, e.g. having a molecular weight of <5000, preferably <3000, more preferred <1500. Preferably the antagonists for use in the present invention are physiologically well acceptable. Auch molecules are typically provided as components of a pharmaceutical composition, optionally including at least one further active ingredient, preferably together with pharmaceutically acceptable excipients and/or additives. Especially preferred CD137 antagonists show a binding constant to either CD137 or CD137 ligand of about at least 10⁷ M⁻¹, more preferred at least 10⁸ M⁻¹, and even more preferred at least 10⁹ M⁻¹.

Antibodies for use in the present invention may be directed against CD137 or CD137 ligand. The term “antibody” comprises polyclonal as well as monoclonal antibodies, chimeric antibodies, humanised antibodies, which may be present in bound or soluble form. Furthermore, an “antibody” according to the present invention may be a fragment or derivative of the afore-mentioned species. Such antibodies or antibody fragments may also be present as recombinant molecules, e.g. as fusion proteins with other (proteinaceous) components. Antibody fragments are typically produced through enzymatic digestion, protein synthesis or by recombinant technologies known to a person skilled in the art. Therefore, antibodies for use in the present invention may be polyclonal, monoclonal, human or humanised or recombinant antibodies or fragments thereof as well as single chain antibodies, e.g. scFv-constructs, or synthetic antibodies.

Polyclonal antibodies are heterogenous mixtures of antibody molecules being produced from sera of animals which have been immunised with the antigen. Subject of the present invention are also polyclonal monospecific antibodies which are obtained by purification of the antibody mixture (e.g. via chromatography over a column carrying peptides of the specific epitope. A monoclonal antibody represents a homogenous population of antibodies specific for a single epitope of the antigen. Monoclonal antibodies can be prepared according to methods described in the prior art (e.g. Köhler und Milstein, Nature, 256, 495-397, (1975); U.S. Pat. No. 4,376,110; Harlow und Lane, Antibodies: A Laboratory Manual, Cold Spring, Harbor Laboratory (1988); Ausubel et al., (eds), 1998, Current Protocols in Molecular Biology, John Wiley & Sons, New York). The disclosure of the mentioned documents is incorporated in total into the present description by reference.

Genetically engineered antibodies for use in the present invention may be produced according to methods as described in the aforementioned references. Briefly, antibody producing cells are cultured to a sufficient optical density, and total RNA is prepared by lysing the cells using guanidinium thiocyanate, acidification with sodium acetate, extraction with phenol, chloroform/isoamyl alcohol, precipitations with mit isopropanol and washing with ethanol. mRNA is typically isolated from the total RNA by chromatography over or batch absorption to oligo-dT-coupled resins (e.g. sepharose). The cDNA is prepared from the mRNA by reverse transcription. The thus obtained cDNA can be inserted into suitable vectors (derived from animals, fungi, bacteria or virus) directly or after genetic manipulation by “site directed mutagenesis” (leading to insertions, inversions, deletions or substitiutions of one or more bases pairs) and expressed in a corresponding host organism. Suitable vectors and host organisms are well known to the person skilled in the art. Vectors derived from bacteria or yeast such as pBR322, pUC18/19, pACYC184, Lambda oder yeast mu vectors may be mentioned as preferred examples. Such vectors are successfully used for cloning the corresponding genes and their expression in bacteria such as E. coli yeast such as Saccharomyces cerevisiae.

Antibodies for use in the present invention can belong to any one of the following classes of immunoglobulins: IgG, IgM, IgE, IgA, GILD and, where applicable, a sub-class of the afore-mentioned classes, e.g. the sub-classes of the IgG class. IgG and ist subclasses, such as IgG1, IgG2, IgG2a, IgG2b, IgG3 or IgGM, are preferred. IgG subtypes IgG1/k or IgG2b/k are especially preferred. A hybridoma clone which produces monoclonal antibodies for use in the present invention can be cultured in vitro, in situ oder in vivo. High titers of monoclonal antibodies are preferably produced in vivo or in situ.

Chimeric antibodies are species containing components of different origin (e.g. antibodies containing a variable region derived from a murine monoclonal antibody, and a constant region derived from a human immunoglobulin). Chimeric antibodies are employed in order to reduce the immunogenicity of the species when administered to the patient and to improve the production yield. For example, in comparison to hybridoma cell lines, murine monoclonal antibodies give higher yiels. However, they lead to a higher immunogenicity in a human patient. Therefore, chimeric human/murine antibodies are preferably used. Even more preferred is a monoclonal antibody in which the hypervariable complementarity defining regions (CDR) of a murine monoclonal antibody are combined with the further antibody regions of a human antibody. Such an antibody is called a humanised antibody. Chimeric antibodies and methods for their production are described in the prior art (Cabilly et al., Proc. Natl. Sci. USA 81: 3273-3277 (1984); Morrison et al. Proc. Natl. Acad. Sci USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci USA 84: 3439-3443 (1987); Sun et al., Proc. Natl. Acad. Sci USA 84: 214218 (1987); Better et al., Science 240: 1041-1043 (1988) und Harlow und Lane, Antibodies: A Laboratory Manual, supra). The disclosure content of the cited documents is incorporated in the present description by reference.

According to the present invention, the term “antibody” comprises complete antibody molecules as well as fragments thereof being capable of binding to CD137 or CD137 ligand, and thus exerting an antagonising effect to CD137 function. Antibody fragments comprise any deleted or derivatised antibody moieties having one or two binding site(s) for the antigen, i.e. one or more epitopes of CD137 or CD137 ligand. Specific examples of such antibody framents are Fv, Fab or F(ab′)₂ fragments or single strand fragments such as scFv. Double stranded fragments such as Fv, Fab or F(ab′)₂ are preferred. Fab und F(ab′)₂ fragments have no Fc fragment contained in intact antibodies. As a beneficial consequence, such fragments are transported faster in the circulatory system and show less non-specific tissue binding in comparison to complete antibody species. Such fragments may be produced from intact antibodies by proteolytic digestion using proteases such as papain (for the production of Fab fragments) or pepsin (for the production of F(ab′)₂ fragments), or chemical oxidation.

Preferably, antibody fragments or antibody constructs are produced through genetic manipulation of the corresponding antibody genes. Recombinant antibody constructs usually comprise single-chain Fv molecules (scFvs, ˜30 kDa in size), in which the V_{H} and V_{L} domains are tethered together via a polypeptide linker to improve expression and folding efficiency. In order to increase functional affinity (avidity) and to increase the size and thereby reduce the blood clearance rates, the monomeric scFv fragments can be complexed into dimers, trimers or larger aggregates using adhesive protein domains or peptide linkers. An example of such a construct of a bivalent scFv dimer is a 60 kDa diabody in which a short, e.g. five-residue, linker between V_{H- and V}_{L}-domains of each scFv prevents alignment of V-domains into a single Fv module and instead results in association of two scFv molecules. Diabodies have two functional antigen-binding sites. The linkers can also be reduced to less than three residues which prevents the formation of a diabody and instead directs three scFv molecules to associate into a trimer (90 kDa triabody) with three functional antigen-binding sites. Association of four scFvs into a tetravalent tetrabody is also possible. Further preferred antibody constructs for use in the present invention are dimers of scFv-CH3 fusion proteins (80 kDa; so-called “minibodies”)

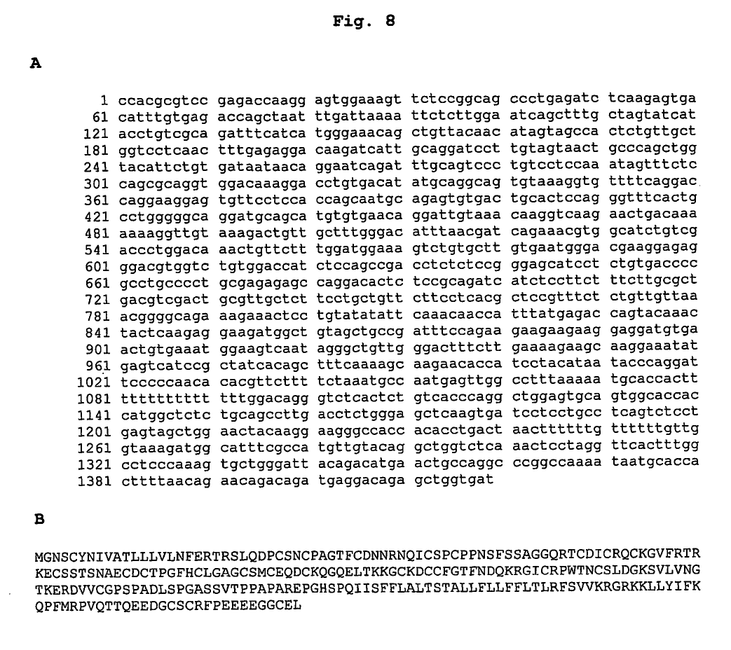

Antibodies for use in the present invention are preferably directed to a peptide or protein which is encoded by a nucleic acid comprising a nucleotide sequence according to GenBank Acc. No. L12964 8 (see FIG. 8A) or a nucleic acid having at least 90%, preferably at least 95%, especially preferred at least 97% homology to the nucleotide sequence according to GenBank Acc. No. L12964.

In particular preferred embodiments of the present invention, antibodies, peptides or small organic molecules are directed to one or more epitope(s) located in the extracellular domain of CD137. Specific examples of antagonistic antibodies against CD137 are clone BBK-2 (Biosource, Ratingen, Germany), clone 4B4-1 (available, e.g., from Ancell or Becton Dickinson) and a polyclonal antibody (anti-4-1BB) available from Chemicon.

Further preferred CD137 antagonists for use according to the present invention are molecules which inhibit the expression of CD137 or CD137 ligand. Specific examples of such species are antisense nucleic acids, especially antisense oligonucleotides, having a sequence being capable of specifically binding to a polynucleotide coding for CD137 or CD137 ligand. According to the present invention, the term “antisense nucleic acid” comprises also peptidic nucleic acids (PNA) which are characterised by a peptide backbone linking the nucleobases. An antisense nucleic acid has a nucleotide sequence which is at least in part complementary to the target sequence, in particular a nucleic acid encoding CD137 or CD137 ligand or a functional fragment or derivative thereof. According to a preferred embodiment, the antisense nucleic acid is at least in part complementary to at least 8, more preferably at least 10, consecutive nucleotides of the human CD137 cDNA sequence according to GenBank Acc. No. L12964, preferably nucleotides 140 to 907 thereof. Further preferred antisense nucleic acids for use in the present invention are part of catalytic nucleic acids such as ribozymes, in particular hammerhead ribozymes, or DNA enzymes, in particular of the type 10-23. A ribozyme is a catalytically active RNA, a DNA enzyme a catalytically active DNA. A further embodiment of an antisense nucleic acid for use in the present invention is a so-called siRNA directed agains CD137 or CD137 ligand. The term “siRNA” means a double-stranded RNA molecule (dsRNA) comprising 19 to 29 bp, preferably 21 to 23 bp, having a nucleotide sequence complementary to the mRNA of CD137 or CD137 ligand. siRNA molecules according to the present invention are commercially available, e.g. from IBA GmbH (Göttingen, Germany).

According to preferred embodiments of the present invention, antisense nucleic acids may be chemically modified, in particular in order to provide a longer half-life in the patient.

Therefore, an “antisense polynukleotide” or “antisense nucleic acid” for use as a CD137 antagonist according to the present invention is a molecule consisting of naturally occuring or modified nucleic acid building blocks, wherein the base sequence or a part thereof is at least complementary to a part of the target sequence, typically the mRNA coding for CD137 or CD137 ligand. Due to its complementarity, the antisense nucleic acid binds (in particular, hybridises) to the target sequence under standard conditions.

Depending on the nucleic acid species, standard hybridisation conditions are represented by temperatures of between about 42 and about 58° C. in an aqueous buffer of between about 0.1 to 5×SSC (1×SSC=0.15 M NaCl, 15 mM sodium citrate, pH 7.2), optionally in the presence of about 50% formamide, e.g. 42° C. in 5×SSC, 50% formamide. Preferred hybridisation conditions for DNA:DANN hybrids are 0.1×SSC at temperatures of between about 20° C. to 45° C., more preferred between about 30° C. to 45° C. Preferred hybridisation conditions for DNA:RNA hybrids are 0.1×SSC at temperatures between about 30° C. to 55° C., more preferred between about 45° C. to 55° C. The hybridisation temperatures given above are examples of melting temperatures calculated for a nucleic acid having a length of about 100 nucleotides and a G+C content of 50% in the absence of formamide. Experimental conditions for DNA hybridisations are described in the prior art (see, e.g., Sambrook et al. “Molecular Cloning”, Cold Spring Harbor Laboratory, 1989) and a person skilled in the art is able to calculate individual conditions in dependence of the length of the nucleic acids, the type of hybrids and the G+C content. Further information about nucleic acid hybridisations is provided by the following references: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Useful antisense nucleic acids in the context of the present invention are typically DNA or RNA species containing or consisting of unmodified or modified nucleotides. Especially in the case of RNA molecules such as antisense RNA and siRNA, it is preferred to incorporate at least one analogue of naturally occurring nucleotides in order to increase the resistance against degradation by RNAses. This is due to the fact that the RNA-degrading enzymes of cells preferably recognise naturally occurring nucleotides Therefore, the degradation of the RNA can successfully be diminished by incorporating nucleotide analogues into the RNA.

The modification of the analogue in comparison to the natural nucleotide may occur at the base as well as at the sugar and/or phosphoric acid moiety of the nucleic acid building block. Specific examples of nucleotide analogues are phosphoroamidates, phosphorothioate, peptide nucleotides (i.e. the antisense nucleic acid is at least in part characterised by a backbone of peptide bonds, thus representing a PNA), methyl phosphonate, 7-deazaguaonsine, 5-methylcytosine and inosine.

As already mentioned above, further antagonists useful in the context of the present invention are antisense expression vectors or corresponding viruses. The antisense construct expressed by the vector or virus may be an antisense sequence of CD137 or CD137 ligand. In such construct at least a part of the cDNA of CD137 or CD137 is cloned into the expression vector or virus in the antisense orientation. Expecially preferred constructs are generated from eukaryotid expression vectors such as pcDNA3 or pRC/RSV (Invitrogen, San Diego, Calif., USA) by inserting the whole or part of the cDNA coding for CD137, e.g. the antisense orientation of the sequence according to GenBank Acc. No. L12964, or C137 ligand into the multiple cloning site of the respective vector. Conveniently, the cDNA or at least a part of it is present as another nucleic acid construct in a suitable cloning vector such as, e.g., Bluescript (Stratagene, San Diego, Calif., USA) or pSPORT. Numerous expression and cloning vectors known to a person skilled in the art are commercially available.

Pharmaceutical compositions or medicaments according to the present invention are especially usefull for treating CD137-expressing tumors. The term “tumor” comprises any kind of cancer or malignancies, including lymphomas, sarcomas, melanomas and carcinomas. Specific examples of CD137-expressing tumors are B cell lymphoma (in particular chronic lymphocytic leukaemia) tumor of the vulva, nephroblastoma, cystadenocarcinoma of the ovary, rhabdomysarcoma, leiomyosarcoma, fibrosarcoma, immunocytoma, non-Hodgkin lymphoma, carcinoma of the portio uteri or basal cell carcinoma.

According to the present invention, one or more CD137 antagonists are typically contained in a composition containing the active ingredient such as polypeptides, nucleic acid constructs, vectors and/or viruses as described above as well as pharmaceutically acceptable excipients, additives and/or carriers (e.g. also solubilisers). Therefore, the present invention discloses a combination of CD137 antagonists as defined above and pharmaceutically acceptable carriers, excipients and/or additives. Corresponding ways of production are disclosed, e.g., in “Remington's Pharmaceutical Sciences” (Mack Pub. Co., Easton, Pa., 1980) which is part of the disclosure of the present invention. As carriers for parenteral administration are disclosed, e.g., sterile water, sterile sodium chloride solutions, polyalkylene glycols, hydrogenated naphthalenes and, in particular biocompatible lactid polymers, lactid/glycolid copolymer or polyoxyethylenelpolyoxypropylene copolymers. Such compositions according to the present invention are envisaged for all medical indications as disclosed above. Moreover, compositions according to the present invention may contain fillers or substances such as lactose, mannitol, substances for covalently linking of polymers such as, for example, polyethylene glycol to inhibitors of the present invention, for complexing with metal ions or for inclusion of materials into or on special preparations of polymer compounds such as, for example, polylactate, polyglycolic acid, hydrogel or onto liposomes, microemulsions, micells, unilamellar or multilamellar vesicles, erythrocyte fragments or spheroplasts. The particular embodiments of the compositions are chosen depending on the physical behaviour, for example with respect to the solubility, stability, bioavailability or degradability. A controlled or constant release of the active substance of the present invention in the composition includes formulations on the basis of lipophilic depots (e.g. fatty acids, waxes or oils). In the context of the present invention are also disclosed coatings of substances or compositions according to the present invention containing such substances, that is to say coatings with polymers (e.g. polyoxamers or polyoxamines). Furthermore, substances or compositions according to the present invention may comprise protective coatings such as protease inhibitors or permeability amplifying agents.

In principle, in the context of the present invention, all administration pathways known in the prior art for substances or compositions according to the present invention are disclosed. Preferably, the preparation of a medicament for the treatment of the tumors mentioned above is carried out via the parenteral, i.e., for example, subcutanous, intramuscular or intravenous, oral or intranasal administration pathway. Typically, pharmaceutical compositions according to the present invention will be solid, liquid or in the form of an aerosol (e.g. spray)—depending on the type of formulation.

Depending on the tumor or cancer to be treated, a local administration of pharmaceutical compositions according to the present invention is envisaged as well. For example, the medicament may be injected directly into the tumor.

According to a preferred embodiment of the present invention, cells can be transfected with a nucleic acid encoding an antagonist according to the present invention and used for the treatment of a CD137-expressing tumor. In this embodiment cells are taken from the patient to be treated, said cells are transfected in vitro with a nucleic acid, e.g. an expression vector encoding a CD137 antagonist, cultured and then transferred into the patient as a retransplant. The transfection is preferably carried out by nucleic acid constructs or expression vectors which combine the expression with a controllable promoter. The transfected endotransplant may be, for example, locally injected—depending on the specific tumor and the specific target cells. A local administration is, in the case of a tumor therapy according to the present invention, preferred. At this, tumor cells are taken from the patient, transfected in vitro and then, if possible, injected directly into the tumor. Further subject matter of the invention relates to the use of CD137 or a functional analogue or derivative thereof for the treatment (or for the preparation of a medicament for the treatment) of conditions characterised by undesired or overactive immune responses.

The term “CD137 functional analogue or derivative thereof” relates to a molecule being capable of exerting the specific immune modulating function of CD137, in particular protection against immune cells such as lymphokine activated killer (LAK) (especially mediated by TGF-β). In a preferred embodiment, the CD137 or functional analogue or derivative is encoded by a nucleic acid comprising a nucleotide sequence having at least 90%, more preferred at least 95%, even more preferred 97% homology to the coding sequence shown in FIG. 8A (nucleotides 140 to 907). Especially preferred for use in the present invention is human CD137 having the amino acid sequence shown in FIG. 8B. Useful for the treatment (or for the preparation of a medicament for the treatment) of characterised by undesired or overactive immune responses are also modified forms of CD137 having one or more amino acid deletions, insertions or substitutions. Particularly useful constructs are recombinant molecules cloned into suitable expression vectors such as pcDNA3 or pRC/RSV. In this context, it is referred to the above description with respect to recombinant DNA technologies.

Due to the bidirectional signalling of CD137 it is also possible to use agonistic anti-CD137 ligand antibodies for the treatment (or for the preparation of a medicament for the treatment) of conditions characterised by undesired or overactive immune responses as well. With respect to preferred antibody constructs it is referred mutatis mutandis to the above general description of CD137 antagonistic antibodies.

Preferred conditions characterised by undesired or overactive immune responses are autoimmune diseases, allergies, asthma and organ transplant rejection.

Therefore, the present invention also discloses a method for treating a patient suffering from a condition as defined above comprising administering an effective amount of the above-defined CD137 functional analogue or derivative thereof and/or an agonistic anti-CD137 ligand antibody.

With respect to suitable components in addition to the active ingredient contained in the medicament or pharmaceutical composition it is referred to the above description for CD137 antagonists. Furthermore, the above description of suitable routes of administration of pharmaceutical composition is also applicable to the CD137 functional analogue or derivative thereof and/or an agonistic anti-CD137 ligand antibody.

THE FIGURES SHOW

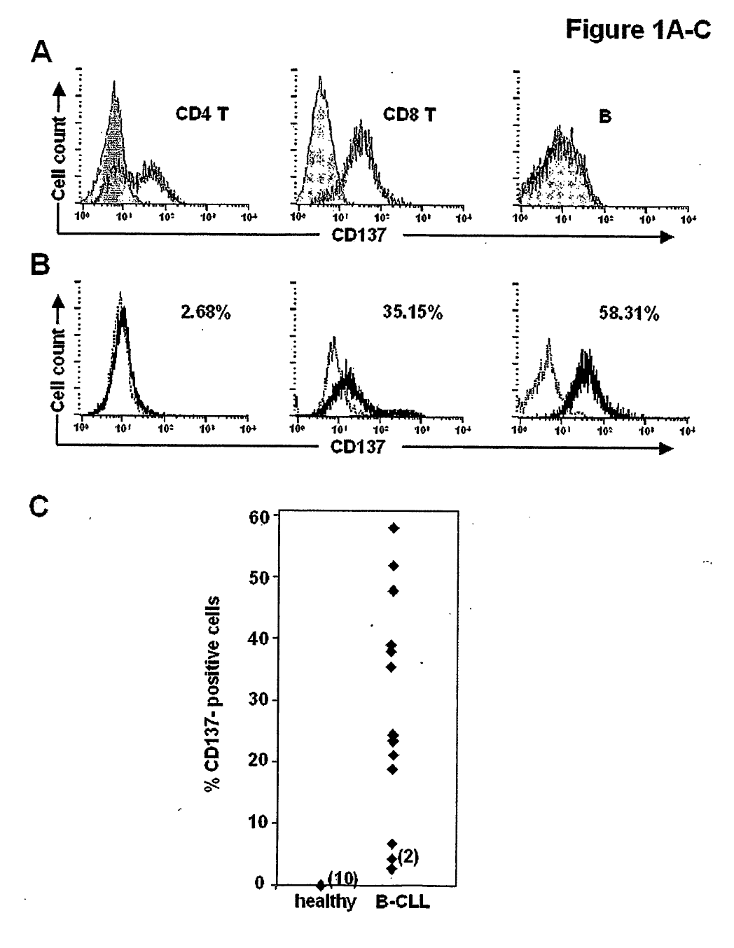

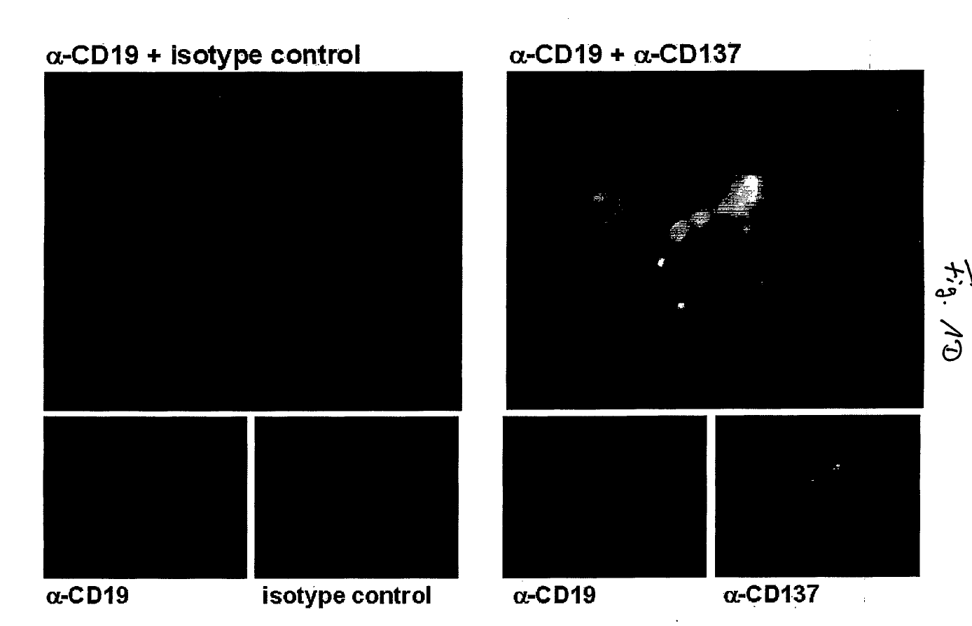

FIG. 1: CD137 is expressed by malignant but not by healthy B cells. (A) 5×10⁶ CD4-positive (CD4) or CD8-positive (CD8) T cells in 1 ml medium were activated by PMA+A23187 for 48 h. B cells (B) were activated by anti-CD40+IL-4. Expression of CD137 was analysed by flow cytometry. Open curve: anti-CD137; filled curve: isotype control. (B) CLL B cells were activated with 10 μg/ml PHA for 24 h and stained with the anti-CD137 antibody BBK-2 (bold line) or and isotype control (dotted line) and analysed by flow cytometry. Shown are examples of a low (left), medium (middle) and high (right) expression of CD137. Indicated are the percentages of CD137-positive CLL cells. (C) Representation of the percentages of CD137-positive B cells from healthy donors and B-CLL patients. The numbers in parenthesis indicate the numbers of samples with an identical result. (D) B-CLL cells were stained with a FITC-labeled anti-CD19 antibody (green) and nuclei were stained with Hoechst 33342 (blue). In addition, the cells were stained with a RPE-labeled antibody for CD137 (red), (right panels), or a RPE-labeled isotype control antibody (left panels). Superimpositions are shown in the top large photographs. Areas of co-localisation of CD19 and CD137 appear orange or yellow. Single stainings for the CD19, CD137 and the isotype control antibody are shown in the smaller photographs beneath the superimpositions. Photographs were taken at a magnification of 400×. (E) Serial frozen sections of a malignant anaplastic B cell lymphoma were stained with antibodies specific for CD137 (CD137), T cells (CD3), B cells (CD20) and an isotype control antibody (control). Staining with hematoxylin and eosin (HE) was used for visualisation of the tissue. Photographs were taken at a magnification of 200×.

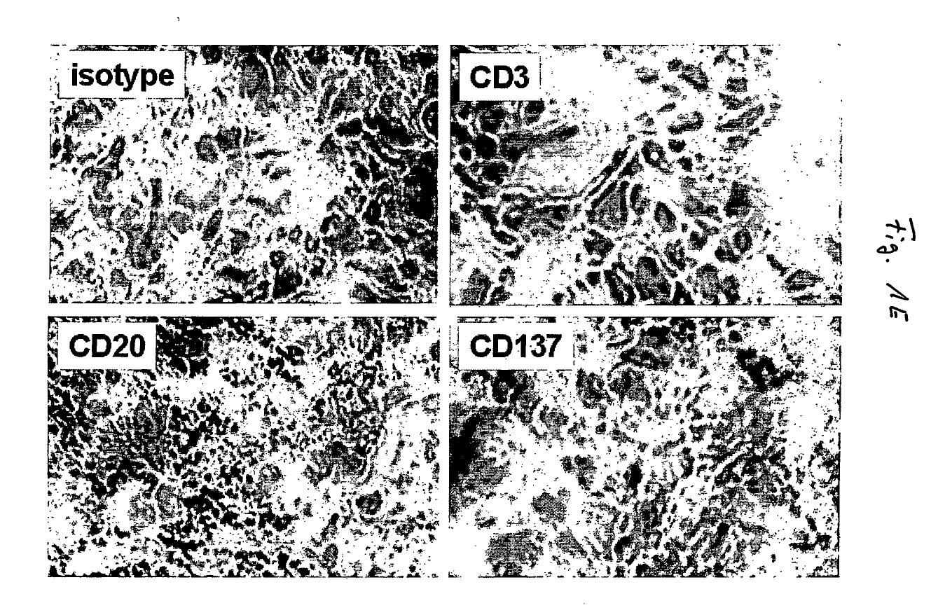

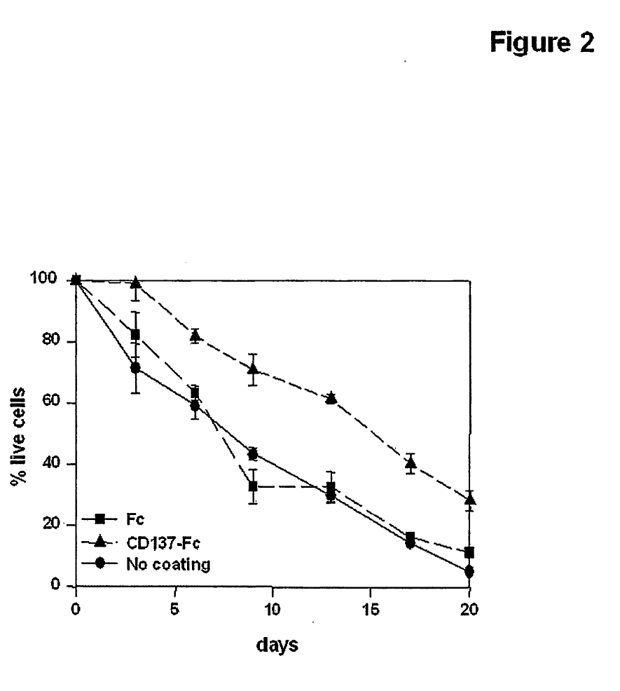

FIG. 2: CD137 extends survival of B-CLL cells. 10⁷ B-CLL cells of patient number 5 were cultured on 5 μg/ml immobilised Fc or CD137-Fc protein. 23% of these CLL cells expressed CD137. The numbers of live cells were determined at day 0, 3, 6, 9, 13, 17 and 20 and by trypan blue staining and are expressed as percentage live cells based on the number of live cells at the beginning of the experiment. Identical results were obtained in six independent experiments.

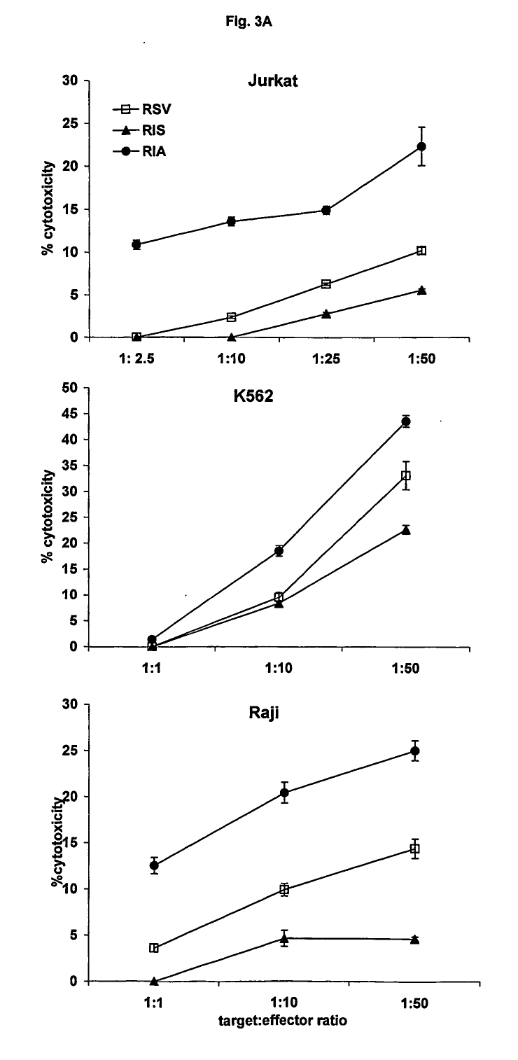

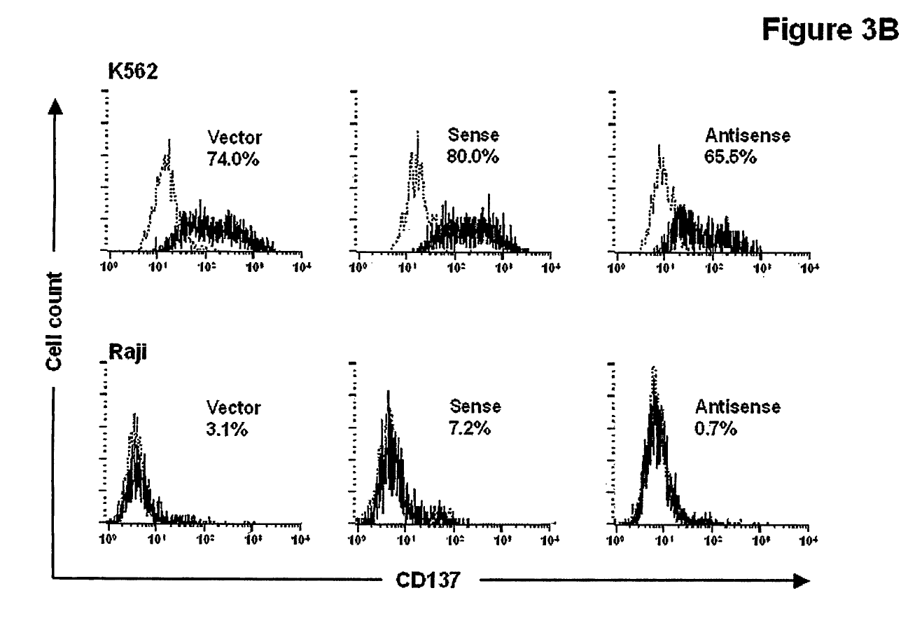

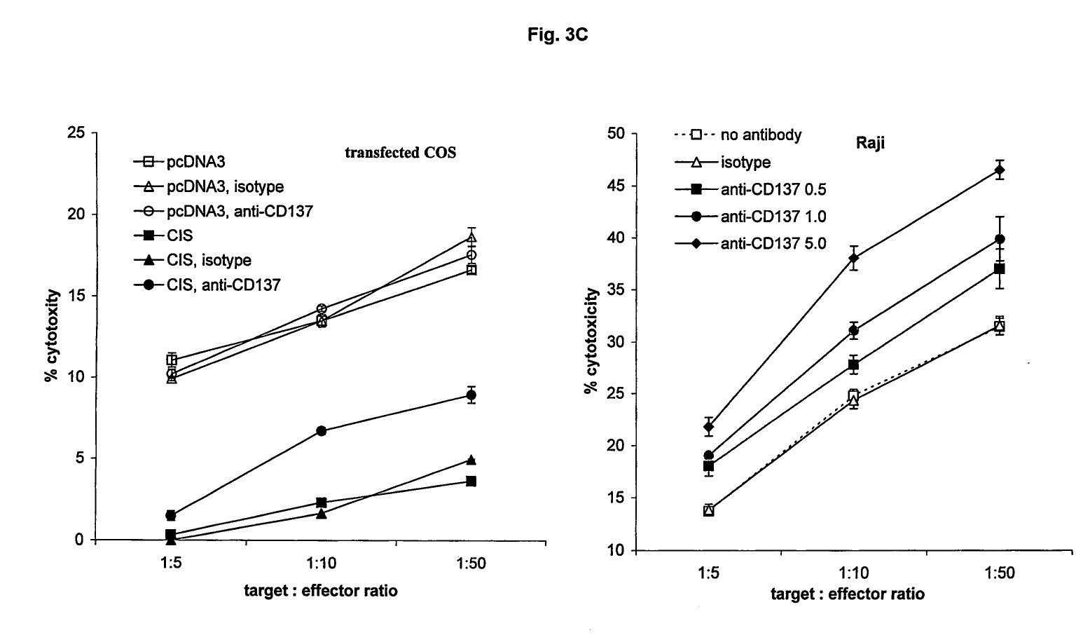

FIG. 3: CD137 protects cells from lysis by LAK cells. (A) PBMC were activated by IL-2 for 3 days to generate LAK cells. Target cells were Jurkat, K562 and Raji cells, which had been transfected with a CD137 expression vector (black triangles), or a CD137 antisense expression vector (black circles), or the empty expression vector (open squares). Each data point represents the mean of 6 independent measurements. Identical results were obtained in three independent experiments. (B) Transfected K562 and Raji cells from (A) were stained for CD137 expression and analysed by flow cytometry. Indicated are the percentages of CD137-positive cells. Vector: empty vector (RSV), Sense: CD137 expression vector (RIS), Antisense: CD137 antisense expression vector (RIA). (C)COS cells were transfected with a CD137 expressin vector (CIS, black symbols) or the empty vector (pcDNA3, open symbols), respectively. No antibody, anti-CD137 antibody (BBK-2) or an isotype control antibody (MOPC21) were added at a concentration of 5 μg/ml 6 h after transfection. The cells were used as targets in a cytotoxicity assay two days later (left panel). Raji cells were grown for 16 h in the presence of 0.5, 1 or 5 μg/ml anti-CD137 antibody (BBK-2) before being used in a cytotoxicity assay. Cultures with no antibody or an isotype control antibody (MOPC21, 5 μg/ml) were used as controls. This experiment was repeated twice with identical results.

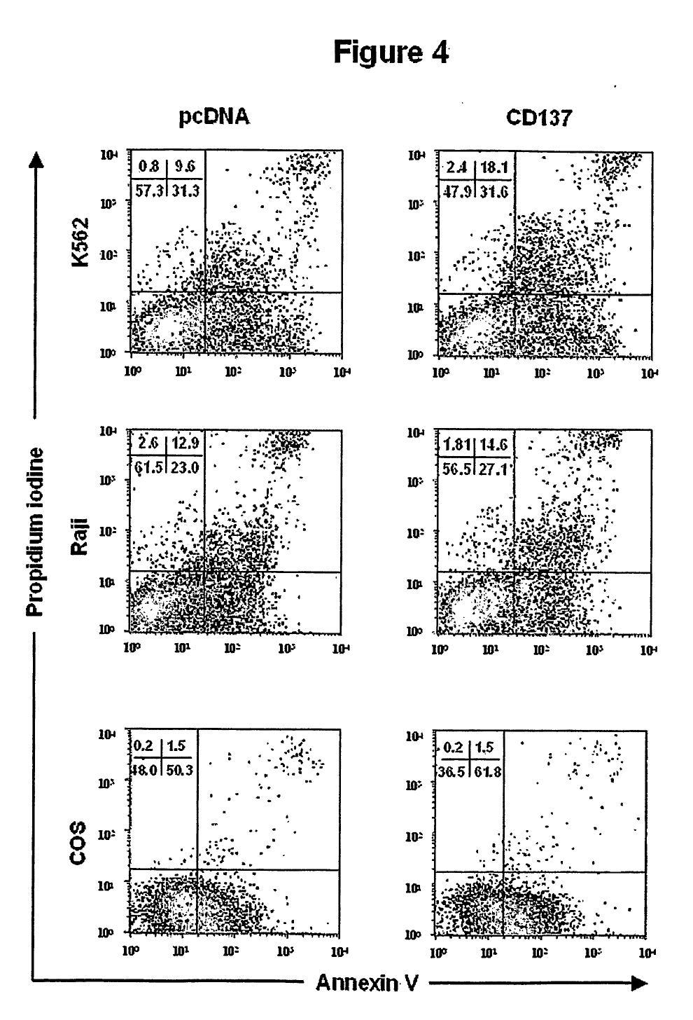

FIG. 4: CD137 induces LAK cell apoptosis. K562 and Raji cells which had been transfected with the CD137 expression vector CIS (CD137), or the empty expression vector (pcDNA3) were used as target cells and LAK cells were used as effector cells. Target and effector cells were incubated for 24 h at a ratio of 1:10. Percentages of live (Annexin V⁻, PE⁻), early apoptotic (Annexin V⁺, PE⁻) and late apoptotic or necrotic effector cells (Annexin V⁺, PE⁺) were determined by flow cytometry and are indicated in the top left corners of the histograms. Comparable results were obtained in three independent experiments.

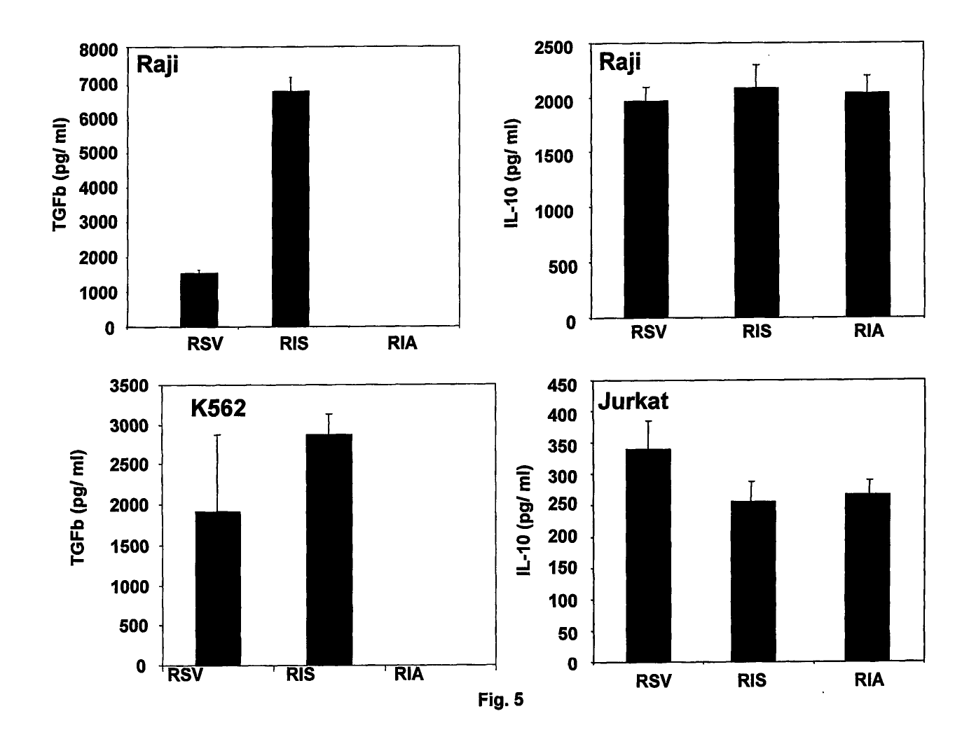

FIG. 5: CD 137 regulates expression of TGF-β by tumor cells. Cells were transfected with a CD137 expression vector (RIS), or a CD137 antisense expression vector (RIA), or the empty vector (RSV). Cytokine concentrations of 24 h supernatants of 106 transfected cells were determined in triplicates. This experiment was repeated four times with similar results.

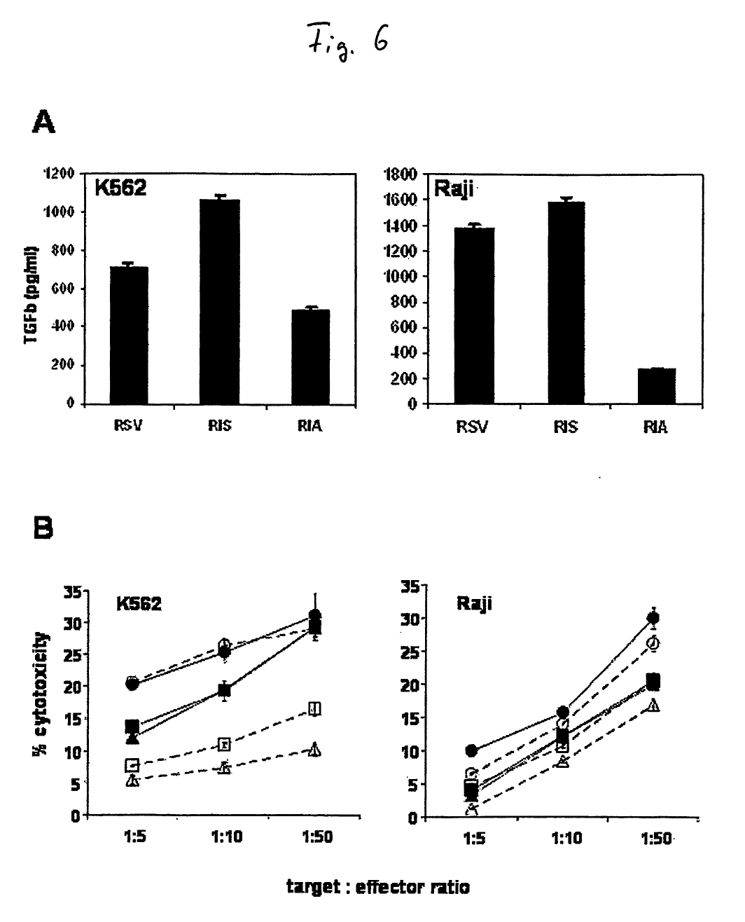

FIG. 6: CD137-induced TGF-β mediates protection from LAK cell lysis. (A) CD137 regulates expression of TGF-β by tumor cells. K562 or Raji cells were transfected with a CD137 expression vector (RIS), or a CD137 antisense expression vector (RIA), or the empty vector (RSV). Cytokine concentrations of 24 h supernatants of 10⁶ transfected cells were determined in triplicates. Depicted are means ± standard deviations. This experiment was repeated four times with comparable results. (B) Neutralisation of TGF-β prevents CD137-mediated protection from LAK cell lysis. K562 or Raji cells were transfected with a CD137 expression vector (triangels), or a CD137 antisense expression vector (circles), or an empty expression vector (squares). Neutralising anti-TGF-β antbody (full symbols), or an isotype control antibody (open symbols) were added to a final concentration of 1 μg/ml. After 2 days the cells were washed and used as target cells in a cytotoxicity assay with LAK cells as effector cells. Each condition was determined in hexaplicates. Identical results were obtained in three independent experiments.

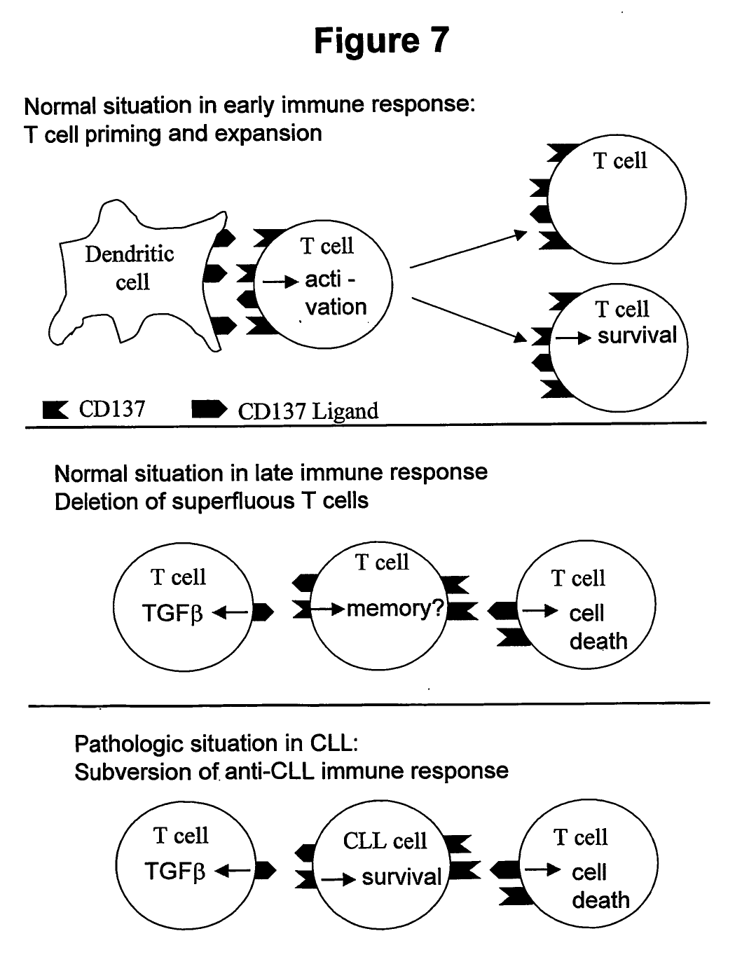

FIG. 7: Schematic representation of activities of CD137 expression on CLL cells. Top panel: During initiation of an immune response dendritic cells present antigen to T cells and provide costimulation via CD137. Middle panel: After the antigen is cleared costimulation of T cells by DC ends. Paracrine induction of inhibitory cytokines and apoptosis in T cells by CD137 becomes predominant and contributes to the termination of the immune response. Bottom panel: CLL cells express CD137 as a neoantigen and utilize its inhibitory activities to downregulate the host anti-tumor immune response.

FIG. 8 cDNA (A) and amino acid sequence (B) of human CD137 (GenBank Acc. No. L12964. The nucleotide sequence coding for the amino acid sequence shown in (B) spans nucleotides 140 to 907 of the cDNA sequence shown in (A).

The following non-limiting examples further illustrate the present invention.

### EXAMPLES

### Example 1

### Materials and Methods

The following materials and methods are used in Examples 2 to 7.

Reagents

The plasmid CMV-ILA-SEN (CIS) was constructed by inserting the human CD137 (ILA) cDNA (cf. FIG. 8A) Into the eukaryotic expression vector pCDNA3 (Invitrogen, San Diego, Calif.). The cDNA was excised from the cloning vector pSPORT by the restriction enzymes EcoRI and HaeIII, which cut in the vector polycloning site 5′ to the CD137 cDNA and in the 3′ untranslated region of the CD137 cDNA at position 921, respectively. The cDNA was inserted into Bluescript (Stratagene, San Diego, Calif.) via EcoRI and SmaI, yielding plasmid ILA-3′del. From there the CD137 cDNA was inserted into pcDNA3 by the restriction sites NotI and HindIII. The plasmids RSV-ILA-SEN (RIS) and RSV-ILA-AS (RIA) are based on the eukaryotic expression vector pRC/RSV (Invitrogen, San Diego, Calif.). The CD137 cDNA fragment from the plasmid ILA-3′del was inserted into pRSV in its sense (RIS) orientation by NotI and HindIII, and in the antisense orientation (RIA) by HindIII, XbaI, respectively. Sequencing confirmed the correct reading frames and sequence of the plasmids. CD137-Fc protein was purified from supernatants of stably transfected CHO cells by protein G sepharose, as described in Schwarz et al. (1996). Human IgG1 Fc protein was purchased from Accurate Chemical and Scientific Corporation, (Westbury, N.Y., USA). Anti-CD137 antibody (clone BBK-2) and its isotype control, MOPC21 were obtained from Biosource (Ratingen, Germany) and Sigma (Deisenhofen, Germany), respectively.

Cells and Cell Culture

Raji and K562 cells were obtained from ECACC (Salisbury, UK). Human peripheral blood mononuclear cells (PBMC) were isolated from fresh blood obtained from healthy volunteers. 50 ml of whole blood were collected and 10 U/ml of heparin were added immediately. The blood was centrifugated for 20 min at 1000 g, the pellet was resuspended in 120 ml of RPMI (without serum) and 5 U/ml of heparine were added. 30 ml of that were layered onto 15 ml of Histopaque (Sigma, Deisenhofen, Germany). After centrifugation at 450 g for 35 min the cells from the boundary layer of each tube were collected and resuspended in 25 ml RPMI, washed in 10 ml RPMI, and finally resuspended at 2-3×10⁶ cells/ml in RPMI 10% FCS. B-CLL cells were isolated from peripheral blood of patients by Histopaque gradient density centrifugation as above. Removal of T cells by negative selection using anti-CD3 beads resulted in 96 to 99% pure B-CLL cell populations. B cells were isolated from PBMC. Fractions with enriched B cells were collected by elutriation and contained between 60% and 80% B cells as estimated by CD19 expression (Andreesen et al., 1990). In a second step using magnetic anti-CD19-beads (Miltenyi, Bergisch-Gladbach) the B cells were purified to >95%.

Cell Proliferation

Proliferation of cell populations was determined in a 96-well microtiter plate. 10⁶ CLL cells per well in a 100 μl volume were pulsed during the last 16 hours of culture with 0.5 μCi ³H-thymidine, harvested and evaluated on the TopCount microplate scintillation counter Packard (Meriden, Conn., USA). Each data point is the mean of five independent measurements and depicted as mean ± standard deviation.

Flow Cytometry Analysis

Cells were analysed using a FACS-Calibur (Becton Dickinson, Mountain View, Calif.) and Celiquest software. 10⁶ cells were used per condition. Cells were washed in fluorescence-activated cell sorting (FACS) buffer (PBS, 2% FCS), resuspended in 50 μl FACS buffer and stained with PE-conjugated anti-CD137 antibody (dilution 1:50, clone 4B4-1; Ancell, Bayport, Minn.), PE-conjugated isotype control antibody (dilution 1.10, Dianova, Hamburg, Germany) and/or PE-conjugated anti-CD19 antibody (dilution 1:25, clone UCHT1, Dako, Hamburg Germany) for 30 min at 4° C. After two washes cells were analysed by flow cytometry.

Confocal Microscopy

Cells were spun onto a microscope slide and dried for 30 min at room temperature, fixed in ice-cold aceton and dried again for 30 min. Thereafter, cells were rehydrated in PBS for 15 min at room temperature and blocked for 30 min with 50 μl PBS, 3% BSA. Double staining was carried out with FITC-conjugated anti-CD19 antibody (dilution 1:10, clone HD37, DAKO, Hamburg, Germany) and biotinylated anti-CD137 antibody (dilution 1:50, clone 4B4-1, Ancell, Bayport, Minn.) for 1 h at 37° C. in the dark. FITC-conjugated murine IgG1 (dilution 1:10, Dianova, Hamburg, Germany) and biotinylated mouse IgG1 (dilution 1:10, Dako, Hamburg, Germany) were used as isotype control antibodies, respectively. After an 1 h incubation at room temperature the slides were washed with PBS and covered with streptavidin-Cy3 for 1 h at room temperature in the dark. After three washes cell nuclei were stained with 4 μg/ml Hoechst 33342 (Sigma, Deisenhofen, Germany) for half an hour at room temperature. The cells were washed and mounted with Mobi Glow (MoBiTec, Goettingen Germany). The slides were stored in the dark at 4° C.

Immunohistochemistry

Frozen tissue sections were fixed with 2% paraformaldehyde for 10 min. Endogenous peroxidases were inactivated by 6.5% hydrogen peroxide in methanol for 15 min. Unspecific staining was blocked by 3% dry milk in PBS for 30 min. 2 μg/ml of anti-CD137 (clone BBK-2, Biosource, Ratingen, Germany) or an isotype control antibody (MOPC 21, Sigma, Deisenhofen, Germany) in 3% dry milk were added overnight. The entire procedure was carried out at RT and after each step the samples were washed three times with PBS. Staining was performed at 37° C. with the ABC kit (Dako, Hamburg, Germany) using diaminobenzidine as substrate. Tissue sections were stained with hematoxylin and embedded in Entellan (Merck, Darmstadt, Germany).

Cytotoxicity Assay

10⁶ target cells per ml were washed twice with PBS, 5% FCS and were loaded with 20 □g/ml of CalceinAM (Molecular Probes, Leiden, The Netherlands) for 20 min at 37° C. Cells were washed twice and 10⁴ loaded target cells per well in 100 μl PBS-F were incubated in 96 well plates with varying numbers of LAK cells for 4 h at 37° C. LAK cells were generated by activating PBMC with IL-2 (100 ng/ml) for 3 days. Values for spontaneous release (FL_{sp.}) were obtained by incubating loaded target cells without LAK cells, and total release (FL_{tot.}) was determined by lysing target cells with lysis buffer (50 mM sodium borate, 0.1% Triton-X 100, pH 9.0). Cells were removed by centrifugation and released Calcein was quantified in a Fluoroscan (Titertek, Fluoroscan II, Meckenheim, Germany) with filter settings at extinction 2, emission 2. The percentage of lysis was calculated according to the following formula: (FL_{assay}−FL_{sp.})/(FL_{tot.}−FL_{sp.})×100=% cytotoxicity.

Apoptosis Assay

Induction of apoptosis was determined by measuring annexin V and propidium iodine staining of cells using the Annexin-V-FLUOS staining Kit (Roche, Mannheim, Germany) according to the manufacture's instructions.

Transfection

K562 and Jurkat cells were transfected using the Lipofectamin/Plus-method (Invitrogen, Groningen, The Netherlands). 10⁶ cells Jurkat or K562 cells were seeded in a 24 well plate in 200 μl serum-free RPMI at. 3 or 4 μg of DNA were diluted in 70 μl of serum-free RPMI for Jurkat or K562 cells, respectively. 5 μl of Plus Reagent were added and the mixture was incubated for 15 min at room temperature. 5 μl of Lipofectamin were added to the mixture and the DNA-Lipofectamin-Plus-Solution was incubated for 15 min at room temperature for complex formation and afterwards added to the cells. 1 ml of RPMI and FCS (10% f.c.) were added 3 h later. Raji cells were transfected by the DMRIE-C-method: Rajis were washed in OPTIMEM (Invitrogen). 3 μl of DMRIE-C (Invitrogen) were diluted in 125 μl OPTIMEM in a 24 well-plate. 0.75 μg DNA diluted in 125 μl OPTIMEM were added and the mixture was incubated for 45 min to allow complex formation. The DMRIE-C-DNA solution was added to 5×10⁵ cells in 50 μl. After a 4 h incubation at 37° C. and 5% CO₂, 1 ml of RPMI/10% FCS were added. Cells were used in experiments two days after transfection.

ELISA

Antibody pairs suitable for IL-10 and TGF-β1 ELISAs were purchased from R&D systems (Wiesbaden, Germany). Buffers were made according to the manufacturer's instructions. 96 well ELISA plates were coated over night with the capture antibody at a 1:180 dilution. The plates were washed three times with washing buffer and blocked with blocking buffer for one hour at 37° C. After three washes, samples and standards were added and incubated for 1 h at 37° C. In the case of TGF-β1, the samples were activated with ⅕ volume of 1 N HCl for 10 min and neutralised with ⅙ volume of 1.2 N NaOH/0.5 M HEPES. IL-10 samples were used without prior treatment. The plates were washed again and incubated with a 1:180 dilution of the detection antibody for 1 h at 37° C. After three washes, a 2 μg/ml ABTS solution in ABTS buffer (Roche Diagnostics, Mannheim, Germany) was added and the plates were analysed in an ELISA reader. Cytokine concentrations were determined In triplicate and are expressed as mean ± standard deviation.

### Example 2

### CD137 is Expressed as a Neoantigen on CLL Cells

Expression of CD137 is inducible in T cells by activation with mitogens and CD137 levels are higher in CD8-positive cells than in CD4-positive ones (FIG. 1A). No expression of CD137 protein could be detected on human peripheral B cells of more than 10 different healthy donors, though several activation conditions were tested, including PHA (10 μg/ml), PMA (5 ng/ml)+calcium ionophore (500 nM), anti-IgM (12.5 μg/ml), and anti-CD40 (10 μg/ml)+IL-4 (100 ng/ml), (FIG. 1A). However, CD137 mRNA has been detected in primary activated B cells, indicating that expression of CD137 in B cells is suppressed at the posttranscriptional level (Schwarz et al., 1995).

B cells isolated from the peripheral blood of B-CLL patients expressed CD137 protein after activation by PHA (10 μg/ml), or PMA (5 ng/ml) plus calcium ionophore (500 nM), (FIG. 1B). CD137 expression was detectable on subsets of CLL B cells from all 14 patients tested, and the numbers of CD137-positve cells ranged from 2.7% to 58.3% (FIG. 1C).

Double staining for CD137 and the B cell-specific marker CD19 and subsequent analysis by confocal microscopy confirmed that the CD137-expressing cells were in fact B cells (FIG. 1D). Co-localisation of CD137 and CD19 on B cells was observed with cells from all of the eight B-CLL patients analysed. Interestingly, in about half of the CD137-positive CLL cells the CD137 protein was not evenly distributed over the cell surface but was localised in clusters which are reminiscent of microdomains (FIG. 1D).

It was necessary to activate the CLL cells with mitogens in order to detect CD137 expression, which raised the question whether CD137 is expressed on malignant B cells in vivo. The CLL cells used were frozen and cultivated in vitro before use. The loss of antigen expression during in vitro cultivation of primary cells is a common phenomenon. Nevertheless, we wanted to verify CD137 expression in vivo and could detect it by immunohistochemistry in a highly malignant anaplastic B cell lymphoma, located on the wall of the sinus cavity (FIG. 1E). The majority of the tumor cells expressed the B cell marker CD20. No T cells could be detected within the tumor based on CD3 staining (FIG. 1E).

### Example 3

### Immobilised CD137 Prolongs Survival of CLL Cells

During the transformation process tumor cells may start to express genes, which are silent in the parental differentiated cells. Many of these neoantigens provide the tumor cells with a growth or selection advantage. The selective expression of CD137 on malignant but not on primary B cells implied a similar role for CD137. Expression of CD137 ligand is constitutively expressed by B cells and upon crosslinking CD137 ligand costimulates B cell proliferation (Pauly et al., 2002). Therefore, CD137 expression could allow CLL cells to enhance their survival and growth in a paracrine manner.

The ability of cell surface-expressed CD137 to crosslink its ligand was simulated by coating a fusion protein consisting of the extracellular domain of CD137 and the constant domain of human immunoglobulin G1 (CD137-Fc) onto cell culture dishes. Untreated and Fc protein coated plates were used as negative controls. Coating was performed with a solution of 10 μg/ml CD137-Fc protein in PBS at 4° C. overnight. Fc protein was used at an equimolar concentration of 5 μg/ml.

Immobilised CD137-Fc significantly prolonged CLL B cell survival while the Fc control protein had no effect (FIG. 2). These data indicate that immobilised CD137 protein crosslinks a ligand or coreceptor on the CLL cells, which delivers the survival signal.

The in vitro survival of CLL cells was donor-dependent and the cells from the six patients which were investigated, had half-lives between 2 and 12 days (Table 1). Most cells were dead after 12-20 days when cultured on uncoated or Fc-coated plates. CLL cells grown on immobilised CD137-Fc survived significantly longer. The maximum effect of CD137 was visible at day 8 when the number of viable cells in all six CLL cell population was 10-30% higher than in the controls. From five out of the six CLL cell populations, cells continued to survive on immobilised CD137-Fc after all cells in the controls had died off. Prolongation of CLL B cell survival by CD137 was statistically significant for cells from all of the 6 patients tested.

TABLE 1
Influence of CD137 on in vitro survival of CLL cells

| CLL patient | Fc | CD137-Fc | x-fold survival | p |
|---|---|---|---|---|
| 1 | 16.0 ± 1.8 | 34.0 ± 9.2 | 2.12 | 0.030 |
| 2 | 12.4 ± 1.0 | 18.8 ± 3.5 | 1.52 | 0.040 |
| 3 | 73.1 ± 6.9 | 84.4 ± 0.9 | 1.16 | 0.043 |
| 4 | 26.4 ± 3.4 | 36.5 ± 1.0 | 1.38 | 0.008 |
| 5 | 32.9 ± 5.6 | 71.0 ± 5.1 | 2.16 | 0.001 |
| 6 | 45.1 ± 1.2 | 71.7 ± 2.3 | 1.58 | >0.001 |
| 10⁷ B cells were cultured on 10 μg/ml immobilised CD137-Fc protein or an equimolar concentration of Fc protein (5 μg/ml). The numbers of live cells were determined on day 8 by trypan blue staining. Four random fields were counted and | | | | |
| # are expressed as percentage of live cells, based on the number of live cells at the beginning of the experiment. The data shown are mean ± standard deviation. “x-fold survival” represents the ration of live cells in CD137-Fc vs. Fc-coated plates. | | | | |

In order to determine a potential effect of CD137 on the growth rate, CLL cells were cultured for 3 or 8 days on immobilised CD137 as described above, and labeled with ³H-thymidine for the last 16 h of culture. In four independent experiments with CLL cells from four different donors up to 2-fold higher proliferation was measured with CD137-Fc treated compared to untreated or Fc-treated CLL cells. However, when incorporated radioactivity was adjusted for the larger number of live cells in the CD137-coated wells, no effect of CD137 on CLL cell proliferation remained.

These data demonstrate that CD137 can prolong the survival of CLL cells but does not influence proliferation. The data further imply that CLL cells express CD137 in order to provide each other with survival signals in a paracrine manner.

### Example 4

### Expression of CD137 Protects Cells from Lysis by Lymphokine Activated Killer (LAK) Cells

It was further investigated whether CD137 expression also influences the host immune response against tumor cells. For assessing the effects of CD137 expression, cells transfected with a CD137 expression vector or the empty control vector were used as target cells in cytotoxicity assays. Since CLL cells were limiting, and more importantly, died during the transfecton procedure we used the Burkitt lymphoma B cell line Raji and the chronic myelogenous leukemia line K562 as targets. Human PBMC were activated for three days with 100 ng/ml of IL-2 and used as lymphokine activated killer (LAK) cells. CD137 transfected Raji or K562 cells were lysed at significantly lower rates than the control transfected cells at target:effector ratios ranging from 1:1 to 1:50 (FIG. 3A).

Since K562 and Raji cells express CD137 constitutively it was possible to perform also the reverse experiment and to test whether reduction of CD137 expression resulted in an increased lysis. K562 and Raji cells which were transfected with a CD137 antisense vector were lysed at higher rates than cells transfected with the empty control vector (FIG. 3A).

Flow cytometric analysis of transfected Raji and K562 cells confirmed that CD137 sense and antisense constructs had indeed changed CD137 expression levels. The expression of CD137 on K562 cells, which have high constitutive CD137 expression, was changed only marginally by transfection. The CD137 sense construct increased the percentage of CD137-positive cells from 74% to 80%, and the antisense construct reduced it from 74% to 65.5% (FIG. 3B). In contrast, Raji cells have low constitutive CD137 expression and therefore the transfection had a much larger effect. In Raji cells the CD137 sense construct more than doubled the percentage of CD137-positive cells, from 3.1% to 7.2%, while the antisense construct reduced it from 3.1% to 0.7% (FIG. 3B). The much larger change of CD137 expression upon transfection in Raji cells compared to K562 cells corresponds well with the larger effect of transfection on Raji cells in the cytotoxicity assay.

In addition to modulating expression of CD137 on target cells, it was also tested whether neutralisation of CD137 by specific antibodies would influence susceptibility to lysis. No constitutive expression is detectable on COS cells with the available anti-CD137 antibodies. Incubation of CD137-transfected COS cells with anti-CD137 antibody for 15 h enhanced lysis of up to threefold, while the antibody did not affect lysis of untransfeected COS cells (FIG. 3C). Pre-incubation with anti-CD137 antibody for 16 h enhanced lysis of Raji cells, which express CD137 constitutively, and enhanced lysis correlated with the concentration of the neutralising anti-CD137 antibody (FIG. 3C).

These experiments involving modulation of CD137 expression and functional inhibition of CD137 by specific antibodies clearly demonstrate that CD137 levels correlate with the protective effect against LAK cell-mediated lysis.

### Example 5

### Induction of Apoptosis by CD137 is not Responsible for Reduced LAK Cytotoxicity

A potential mechanism for protection against lysis by LAK cells could be induction of cell death. CD137 has been shown previously to induce apoptosis in T cells (Schwarz et al., 1996; Michel et al., 1999).

In order to investigate whether LAK cells are being driven into apoptosis by CD137-expressing target cells, LAK cells were co-cultured with CD137-transfected and mock-transfected Raji or K562 cells. 24 h later the LAK cells were analysed for signs of apoptosis and necrosis using propidium iodine and annexinV staining. Compared to control cells CD137-transfected Raji and K562 cells increased LAK cell death (FIG. 4). The percentage of late apoptotic or necrotic LAK cells (Annexin⁺, PE⁺) rose from 9.6% to 18.1% in the case of CD137-transfected K562 cells. CD137 transfection of Raji cells increased the percentage of late apoptotic or necrotic LAK cells (Annexin⁺, PE⁺) from 12.9% to 14.6%, and the percentage of early apoptotic (Annexin⁺, PE⁻) LAK cells from 23% to 27.1%.

In order to investigate whether LAK cells are being driven into apoptosis by CD137-expressing target cells, LAK cells were co-cultured with CD137-transfected and mock-transfected Raji or K562 cells. 24 h later the LAK cells were analysed for signs of apoptosis and necrosis using propidium iodine and annexinV staining. Compared to control cells CD137-transfected Raji and K562 cells increased LAK cell death (FIG. 4). The percentage of late apoptotic or necrotic LAK cells (Annexin⁺, PE⁺) rose from 9.6% to 18.1% in the case of CD137-transfected K562 cells. CD137 transfection of Raji cells increased the percentage of late apoptotic or necrotic LAK cells (Annexin⁺, PE⁺) from 12.9% to 14.6%, and the percentage of early apoptotic (Annexin⁺, PE⁻) LAK cells from 23% to 27.1% (Table 2).

TABLE 2
Influence of CD137 on LAK cell death

| | | live | early apoptotic | late apoptotic | necrotic |
|---|---|---|---|---|---|
| COS cells | target: | AnnexinV⁻ | Annexin V⁺ | Annexin V⁺ | Annexin V⁻ |
| transfected with: | effector ratio | PE⁻ | PE⁻ | PE⁺ | PE⁺ |
| — | — | 75.2 | 6.2 | 16.6 | 2.0 |
| PcDNA3 | 1:1 | 70.3 | 9.4 | 17.5 | 2.8 |
| | 1:2.5 | 76.6 | 5.9 | 15.4 | 2.2 |
| | 1:10 | 77.8 | 5.5 | 14.9 | 1.9 |
| CD137 | 1:1 | 69.8 | 7.1 | 19.9 | 3.2 |
| | 1:2.5 | 71.1 | 6.2 | 18.7 | 4.0 |
| | 1:10 | 74.3 | 5.5 | 16.8 | 3.5 |
| PBMC were activated by 100 ng/ml of IL-2 for 3 days to generate LAK cells. COS cells which had been transfected with the CD137 expression vector RIS (CD137), or the empty expression vector (pcDNA3) were used as target cells. Effector and target cells were incubated for 24 h at | | | | | |
| # indicated ratios. Percentages of live (AnnexinV−, PE−), early apoptotic (Annexin+, PE−), late apoptotic (AnnexinV+, PE+) and necrotic effector cells (AnnexinV−, PE+) were determined by flow cytometry. Identical results were obtained in two independent experiments. | | | | | |

Since Raji and K562 cells express CD137 constitutively. Therefore, the experiment using COS cells, which do not express CD137, was repeated in order to exclude any potential interference of constitutive CD137. LAK cells which were incubated with CD137-transfected COS cells displayed a higher rate of apoptosis compared to LAK cells which were incubated with mock-transfected COS cells, as evidenced by the higher percentage of apoptotic (Annexin⁺, PE⁻) cells (61.8% vs. 50.3%), (FIG. 4).

These data demonstrate that CD137 expression enables cells to induce apoptosis in immune cells. Therefore, the expression of CD137 as a neoantigen is used by tumor cells as a mechanism for escaping the patient's immune response.

### Example 6

### CD137 Induces Expression of TGF-β

As shown above, the extent of induction of apoptosis by CD137 was overall small. And the change in LAK cell apoptosis after transfecting the target cells with CD137 sense or antisense vectors was smaller that the change in target cytotoxicity. Further, CD137-induced apoptosis is a slow process peaking around day 3 (Schwarz et al., 1996; Michel at al., 1999) while the cytotoxicity assay lasted only 4 h. Therefore, these data suggest that induction of apoptosis in LAK cells is not the sole mechanism of CD137-mediated protection.

Besides induction of apoptosis in immune cells, secretion of antiinflammatory cytokines is a powerful mechanism of tumor cells to evade immune surveillance. The antiinflammatory activities of IL-10 and TGF-β and their utilisation by tumor cells are well documented (Akhurst and Derynck, 2001; Pasche, 2001). Therefore, concentrations of TGF-β and IL-10 were measured in supernatants of LAK cells after exposure to CD137. Co-culture of CD137 expressing cells had no detectable effect on TGF-β or IL-10 secretion by LAK cells.

However, CD137 regulated cytokine expression by the target cells. Raji cells express TGF-β and IL-10 constitutively. Transfection with the CD137 expression vector slightly increased levels of TGF-β, (from 1380±31 to 1579±45 μg/ml), while transfection with the CD137 antisense vector significantly reduced TGF-□ secretion (to 270±11 μg/ml) by Raji cells (FIG. 6A). Levels of IL-10 remained unchanged (not shown). K562 cells also express TGF-β constitutively but no IL-10 was detectable in their supernatants. Similarly to Raji cells, levels of TGF-β were increased by CD137 sense (from 710±24 to 1060±26 μg/ml) transfection and decreased by CD137 antisense transfection (to 490±13 μg/ml), respectively (FIG. 6A). These experiments demonstrate that TGF-β is induced by CD137 and that TGF-β levels correlate with the amount of CD137. IL-10 secretion seemed to be independent of CD137.

### Example 7

### CD137-Induced TGF-β Mediates Protection Against Lysis by LAK Cells

According to the present invention, it has been demonstrated that the amount of CD137 expression by the target cells correlated (1) with the degree of protection from LAK-mediated cytotoxicity, and (2) with TGF-β levels secreted by the target cells. Therefore, it is reasonable to assume that TGF-β induction contributes to CD137-mediated protection. In order to verify this hypothesis neutralising anti-TGF-β antibodies were added to the target cells during the period from 6 h after transfection up to the cytotoxicity assay at day 2. Neutralisation of TGF-β during this 2-day period prior to the cytotoxicity assay rendered the target cells more susceptible to LAK lysis (FIG. 6B). Target cells were thoroughly washed before being used in the cytotoxicity assay to avoid carry-over of anti-TGF-β antibodies into the assay. Also, no TGF-β could be detected in the supernatants of the cytotoxicity assay at the end of the experiment. Further, neutralising anti-TGF-β antibodies had no effect when added directly into the cytotoxicity assay, instead of being added to the target cells prior to the assay. This indicates that TGF-β does not mediate protection from lysis by inhibiting LAK cell activity. Rather, it seems that CD137-induced TGF-β secretion makes target cells more resistant to lysis by LAK cells. A possible mechanism would be induction of members of the bcl-2 family, which have been widely documented to raise the threshold of cell death induction (Adams and Cory; 1998).

Tumor cells express neoantigens as a result of random mutations. Some of these neoantigens provide the tumor cells with growth and/or survival advantages and become selected and enriched in the tumor cell population.

The present invention is based on the finding that immobilised CD137 protein prolongs B-CLL cell survival in vitro. This effect was observed with cells from all six patients tested. No effect of CD137 on CLL cell proliferation could be observed.

Crosslinking of CD137 ligand through immobilised CD137 protein or CD137 expressed on transfected cells enhances proliferation and immunoglobulin synthesis of primary B cells (Pollok et al., 1994; Pauly et al., 2002). Under physiological conditions this co-stimulation would occur during interactions of T cells with B cells or FDC (DeBenedette et al., 1997; Pauly et al., 2002). The ectopic expression of CD137 could enable malignant B cells to imitate these interactions and to provide each other mutually with survival signals in a paracrine manner. Functional signaling of CD137 is implied by its clustering into cell surface structures, which are compatible with microdomains. Similar clustering or assembly to rafts has been observed for other costimulatory molecules on immune cells such as CD28 and LFA-1 Grakoui et al., 1999; Malissen et al., 1999).

Reverse signaling through a CD137 ligand also occurs in monocytes. Immobilised but not soluble CD137 protein induces activation and proliferation and prolongs survival of peripheral monocytes (Langstein et al., 1998; Langstein et al., 1999a; Langstein et al., 1999b; Langstein et al., 2000). These data suggest that activation through a CD137 ligand also occurs in other APC and may be a common feature of APC.

Ectopic expression of CD137 provided target cells with protection from lysis by LAK cells while reduction of constitutive CD137 expression enhanced susceptibility. The protective effect of CD137 was further confirmed using neutralising anti-CD137 antibodies, which enhanced lysis by LAK cells.

FIG. 5 illustrates a possible mechanism of the physiological role of CD137 and its utilisation by malignant B cells. During the initial phase of an immune response antigen-specific T cells start to express CD137 after TCR engagement. CD137 ligand expressed by APC crosslinks CD137 delivering further activating signals to T cells (FIG. 5, upper panel). Costimulation by CD137 ligand or agonistic anti-CD137 antibodies enhances T cell activity in vitro and in vivo enabling tumor eradication in mice (Pollok et al., 1993; Schwarz et al., 1996; Melero et al., 1997). In the late phase of an immune response when the pathogen or antigen is cleared, APC will no longer provide growth and survival signals and the inhibitory activities of CD137 may gain the upper hand (FIG. 5, middle panel). Ectopic CD137 expression enables malignant B cells to use these mechanisms to defend themselves against infiltrating cytotoxic T cells (FIG. 5, lower panel). Though antigen-primed APC should be present in tumor patients and deliver survival signals to the tumor-specific cytotoxic T cells, the final outcome may be determined by the relative amounts of CD137 ligand on APC versus CD137 on malignant B cells.

According to the present invention, it has been shown that through the ectopic expression of CD137, malignant B cells acquire the capability to inhibit LAK cell cytotoxicity via induction of apoptosis. This helps the tumor cells to escape from the host immune surveillance. The malignant B cells retain the ability of primary B cells to become activated through CD137 ligand. This activity provides the basis for prolonged CD137-mediated CLL B cell survival. Therefore, interference with CD137 function by corresponding antagonists, e.g. by down regulation of CD 137 levels or neutralisation of CD137, provides a powerful means for therapy of CD137-expressing tumors.

### REFERENCES

Alderson, M. R., Smith, C. A., Tough, T. W., Davis-Smith, T., Armitage, R. J., Falk, B., Roux, E., Baker, E., Sutherland, G. R., Din, W. S., Goodwin, R. G. (1994) Molecular and biological characterization of human 4-1BB and its ligand. Eur. J. Immunol. 24, 2219-2227.
Andreesen, R., Brugger, W., Scheibenbogen, C., Kreutz, M., Leser, H. G., Rehm, A., Lohr, G. W. (1990) Surface phenotype analysis of human monocyte to macrophage maturation. J. Leukoc. Biol. 47, 490-497.
Boussaud V, Soler, P., Moreau, J., Goodwin, R. G., Hance, A. J. (1998) Expression of the three member of the TNF-R family of receptors (4-1BB, lymphotoxin-β receptor, and Fas) in human lung. Eur. Resp. J. 12, 926-931.
DeBenedette, M. A., Chu, N. R., Pollok, K. E., Hurtado, J., Wade, W. F., Kwon, B. S., Watts, T. H. (1995) Role of 4-1BB ligand in costimulation of T lymphocyte growth and its upregulation on M12 B lymphomas by CAMP. J. Exp. Med. 181, 985-992.
DeBenedette, M. A., Shahinian, A., Mak, T. W., Watts, T. H. (1997) Costimulation of CD28-T lymphocytes by 4-1BB ligand. J. Immunol. 158, 551-559.
Desbarats, J., Duke. R. C., Newell, M. K. (1998) Newly discovered role for Fas ligand in the cell-cycle arrest of CD4+ T cells. Nat. Med. 4, 1377-1382.
Grakoui, A., Bromley, S. K., Sumen, C., Davis, M. M., Shaw, A. S., Allen, P M., Dustin, M. L. (1999) The immunological synapse: a molecular machine controlling T cell activation. Science. 285, 221-227.
Goodwin, R. G., W. S. Din, T. Davis-Smith, D. M. Anderson, S. D. Gimpel, T. A. Sato, C. R. Maliszewski, C. I. Brannan, N. G. Copeland, N. A. Jenkins, et-al. 1993. Molecular cloning of a ligand for the inducible T cell gene 4-1BB: a member of an emerging family of cytokines with homology to tumor necrosis factor. Eur. J. Immunol. 10:2631.
Hurtado, J. C., Kim, S. H., Pollok, K. E., Lee, Z. H., Kwon, B. S. (1995) Potential role of 4-1BB in T cell activation. Comparison with the costimulatory molecule CD28. J. Immunol. 155, 3360-3367.
Jachimczak, P., Bogdahn, U., Schneider, J., Behl, C., Meixensberger, J., Apfel, R., Dorries, R., Schlingensiepen, K. H., Brysch, W. (1993) The effect of transforming growth factor-beta 2-specific phosphorothioate-anti-sense oligodeoxynucleotides in reversing cellular immunosuppression in malignant glioma. J. Neurosurg. 78, 944-951.
Kempis J. von, Schwarz, H., Lotz. M. (1997) Differentiation-dependent and stimulus-specific expression of ILA, the human 4-1BB-homologue, in cells of mesenchymal origin. Osteoarthritis and Cartilage. 5, 394-406.
Kwon, B. S., Weissman, S. M. (1989) cDNA sequences of two inducible T-cell genes. Proc. Natl. Acad. Sci. USA. 86, 1963-1967.
Kwon, B. S., Hurtado, J. C., Lee, Z. H., Kwack, K. B., Seo, S. K., Choi, B. K., Koller, B. H., Wolisi, G., Broxmeyer, H. E., Vinay, D. S. (2002) Immune responses in 4-1BB (CD137)-deficient mice. J. Immunol. 168, 5483-5490.
Langstein J., Michel, J., Fritsche, J., Kreutz, M., Andreesen, R., Schwarz, H. (1998) CD137, (ILA/4-1BB), a member of the TNF receptor family regulates monocyte activation via reverse signaling. J. Immunol. 160, 2488-2494.
Langstein, J., Schwarz, H. (1999) Identification of CD137 as a potent monocyte survival factor. J. Leuk. Biol. 65, 829-833.
Langstein, J., Michel, J., Schwarz, H. (1999) CD137 Induces Proliferation and Endomitosis in Monocytes. Blood 94, 3161-3168.
Langstein, J., Becke, F. M., Soellner, L., Krause, G., Brockhoff, G., Kreutz, M., Andreesen, R., Schwarz, H. (2000) Comparative analysis of CD137 and LPS effects on monocyte activation, survival, and proliferation. Biochem. Biophys. Res. Corn. 273,117-122.
Lichtenfels, R., Biddison, W. E., Schulz, H., Vogt, A. B., Martin, R. (1994) CARE-LASS (calcein-release-assay), an improved fluorescence-based test system to measure cytotoxic T lymphocyte activity. J. Immunol. Meth. 172, 227-239.
Malissen, B. (1999) Dancing the Immunological two-step. Science. 285, 207-208.
Suzuki, I., Fink, P. J. (1998) Maximal proliferation of cytotoxic T lymphocytes requires reverse signaling through Fas ligand. J. Exp. Med. 187,123-128.
Martinet, O., Ermekova, V., Qiao, J. Q., Sauter, B., Mandeli, J., Chen, L., Chen, S. H. (2000) Immunomodulatory gene therapy with interleukin 12 and 4-1BB ligand: long-term remission of liver metastases in a mouse model. J. Natl. Cancer Inst. 92, 931-936.
Melero, I., Shuford W. W., Newby, S. A., Aruffo, A., Ledbetter, J. A., Hellströam, K. E., Mittler, R. S., Chen, L. (1997) Monoclonal antibodies against 4-1BB T cell activation molecule eradicate established tumors. Nat. Med. 3, 682-685.
Melero I, Bach N, Hellstrom K E, Aruffo A, Mittler R S, Chen L (1998) Amplification of tumor immunity by gene transfer of the co-stimulatory 4-1BB ligand: synergy with the CD28 co-stimulatory pathway. Eur J Immunol 28: 1116-1121.
Michel, J., Langstein, J., Krammer, P., Schwarz, H. (1999) CD137-induced apoptosis is independent of CD95. Immunology 98, 42-46.
Michel, J., Langstein, J., Hofstädter, F., Schwarz, H. (1998) A soluble form of CD137 (ILA/4-1BB) is released by activated lymphocytes and is detectable in sera of patients with rheumatoid arthritis. Eur. J. Immunol. 28, 290-295.
Michel, J., Schwarz, H. (2000) Soluble CD137 is selectively expressed by T lymphocytes and correlates with activation induced cell death of lymphocytes. Cytokine 12, 742-746.
O'Connell, J., Bennett, M. W., O'Sullivan, G. C., Collins, J. K., Shanahan, F. (1999) The Fas counterattack: cancer as a site of immune privilege. Immunol. Today. 20, 46-52.
Pauly, S., Broll, K., Giegerich, G., Schwarz, H. (2002) CD137 regulates B lymphocyte proliferation and differentiation in germinal centers. J. Leuk. Biol. 72, 35-42.
Pollok, K E., Kim, Y. J., Zhou, Z., Hurtado, J., Kim, K. K., Pickard, R. T., Kwon, B. S. (1993) Inducible T cell antigen 4-1BB. Analysis of expression and function. J. Immunol-150, 771-7781.
Pollok, K. E., Kim, Y. J., Hurtado, J., Zhou, Z., Kim, K. K., Kwon, B. S. (1994) 4-1BB T-cell antigen binds to mature B cells and macrophages, and costimulates anti-mu-primed prime splenic B cells. Eur. J. Immunol. 24, 367-374.
Schwarz, H., Tuckwell, J., Lotz, M. (1993) A receptor induced by lymphocyte activation ILA: a new member of the human nerve growth factor/tumor necrosis factor receptor family. Gene 134, 295-298.
Schwarz, H., Valbracht, J., Tuckwell, J., Kempis, J., Lotz, M. (1995) ILA, the human 4-1BB homologue is inducible in lymphoid and other cell lines. Blood 85, 1043-1052.
Schwarz, H., Blanco, F., Valbracht, J., Kempis. J., Lotz, M. (1996) ILA, A member of the human NGF/TNF receptor family regulates T lymphocyte proliferation and survival. Blood 87, 2839-2845.
Schwarz, H., K. Arden, and M. Lotz. 1997. CD137, a Member of the Tumor Necrosis Factor Receptor Family is Located on Chromosome 1p36, in a Cluster of Related Genes, and Colocalizes with Several Malignancies. Biochem. Biophys. Res. Comm. 235(3):699.
Shuford, W. W., Klussman, K., Tritchier, D. D., Loo, D. T., Chalupny, J., Siadak, A. W., Brown, T. J., Emswiler, J., Raecho, H., Larsen, C. P., Pearson, T. C., Ledbetter, J. A., Aruffo, A., Mittler, R. S. (1997) 4-1BB costimulatory signals preferentially induce CD8+ T cell proliferation and lead to the amplification in vivo of cytotoxic T cell responses. J. Exp. Med. 186, 47-55.
Strand, S., Hofmann, W. J., Hug, H., Muller, M., Otto, G., Strand, D., Mariani, S. M., Stremmel, W., Krammer, P. H., Galle, P. R. (1996) Lymphocyte apoptosis induced by CD95 (APO-1/Fas) ligand-expressing tumor cells—a mechanism of immune evasion? Nat. Med. 2, 1361-1366.
Tan, J. T., Whitmire, J. K., Ahmed, R., Pearson, T. C., Larsen, C. P. (1999) 4-1BB ligand, a member of the TNF family, is important for the generation of antiviral CD8 T cell responses. J. Immunol. 163, 4859-4868.
Ye, Z., Hellstrom, I. Hayden-Ledbetter, M., Dahlin, A., Ledbetter, J. A., Hellstrom, K. E. (2002) Gene therapy for cancer using single-chain Fv fragments specific for 4-1 BB. Nat. Med. 8, 343-348.

1. Use of a CD137 antagonist for the preparation of a medicament for the treatment of CD137-expressing tumors.

2. Use according to claim 1 wherein the CD137 antagonist is selected from the group consisting of a CD137-specific antibody, peptide, organic small molecule, antisense oligonuclotide, siRNA, antisense expression vector or recombinant virus.

3. Use according to claim 1 wherein the antibody is directed to at least one epitope of the amino acid sequence of human CD137 shown in FIG. 8B.

4. Use according to claim 3 wherein the CD137-specific antibody is clone BBK-2 or clone 4B4-1.

5. Use according to claim 2 wherein the CD137-specific antisense expression vector is RSV-ILA-AS.

6. Use according to claim 1 wherein the CD137 antagonist is selected from the group consisting of a CD137 ligand-specific antibody, peptide, organic small molecule, antisense oligonucleotide, siRNA, antisense expression vector or recombinant virus.

7. Use according to claim 1 wherein the tumor is a B cell lymphoma, tumor of the vulva, nephroblastoma, cystadenocarcinoma of the ovary, rhabdomysarcoma, leiomyosarcoma, fibrosarcoma, immunocytoma, non-Hodgkin lymphoma, carcinoma of the portio uteri or basal cell carcinoma.

8. Use according to claim 7 wherein the B cell lymphoma is chronic lymphocytic leukaemia.

9. Method of treating a tumor patient comprising administering an effective amount of a CD137 antagonist.

10. Method according to claim 9 wherein the CD137 antagonist is selected from the group consisting of a CD137-specific antibody, peptide, organic small molecule, antisense oligonuclotide, siRNA, antisense expression vector or recombinant virus.

11. Method according to claim 9 wherein the tumor is a B cell lymphoma, tumor of the vulva, nephroblastoma, cystadenocarcinoma of the ovary, rhabdomysarcoma, leiomyosarcoma, fibrosarcoma, immunocytoma, nonhodgkin lymphoma, carcinoma of the portio uteri or basal cell carcinoma.

12. Method according to claim 11 wherein the B cell lymphoma is chronic lymphocytic leukaemia.

13. Use of CD137 or a functional analogue or derivative thereof for the preparation of a medicament for the treatment of conditions characterised by undesired or overactive immune responses.

14. Use according to claim 13 wherein the CD137 or functional analogue or derivative is encoded by a nucleic acid comprising a nucleotide sequence having at least 90% homology to the coding sequence shown in FIG. 8A.

15. Use according to claim 14 wherein the CD137 has the amino acid sequence shown in FIG. 8B.

16. Use according to claim 13 wherein the condition is selected from autoimmune diseases, allergies, asthma and organ transplant rejection.

17. Use of an agonistic anti-CD137 ligand antibody for the preparation of a medicament for the treatment of conditions characterised by undesired or overactive immune responses.

18. Use according to claim 17 wherein the condition is selected from autoimmune diseases, allergies, asthma and organ transplant rejection.

19. Method for treating a patient suffering from a condition characterised by undesired or overactive immune responses comprising administering an effective amount of CD137 or a functional analogue or derivative thereof and/or an agonistic anti-CD137 ligand antibody.

20. Method of claim 19 wherein the CD137 or functional analogue or derivative thereof is encoded by a nucleic acid comprising a nucleotide sequence having at least 90% homology to the coding sequence shown in FIG. 8A.

21. Method of claim 19 wherein the condition is selected from autoimmune diseases, allergies, asthma and organ transplant rejection.

22. Method according to claim 9 wherein the CD137 antagonist is an antibody directed to at least one epitope of the amino acid sequence of human CD137 shown in FIG. 8B.

23. Method according to claim 9 wherein the CD137 antagonist is clone BBK-2 or clone 4B4-1

24. Method according to claim 9 wherein the CD137 antagonist is the antisense expression vector RSV-ILA-AS.

25. Method according to claim 9 wherein the CD137 antagonist is selected from the group consisting of a CD137 ligand-specific antibody, peptide, organic small molecule, anti sense oligonucleotide, siRNA, anti sense expression vector or recombinant virus.

26. Method of claim 20 wherein the CD137 has the amino acid sequence shown in FIG. 8B.
